(19) United States
(12) Patent Application Publication
(10) Pub. No.: US 2020/0061365 A1
(43) Pub. Date: Feb. 27, 2020

(54) CELL STORAGE CONTAINER FOR USE WITH STERILE DOCKING SYSTEMS

(71) Applicant: Instant Systems, Inc., Norfolk, VA (US)

(72) Inventors: Madeline C. ALDEN, Norfolk, VA (US); Kimber L. BURLEY, Fulton, MD (US); Tara C. RAMSEY, Norfolk, VA (US)

(73) Assignee: Instant Systems, Inc., Norfolk, VA (US)

(21) Appl. No.: 16/549,732

(22) Filed: Aug. 23, 2019

Related U.S. Application Data
(60) Provisional application No. 62/722,810, filed on Aug. 24, 2018.

(51) Int. Cl. A61M 39/18; A61J 1/14

(52) U.S. Cl. CPC A61M 39/18; A61M 2039/1077; A61J 1/1468

(57) ABSTRACT

An apparatus includes a container, a connector, and a tube. The container defines a storage volume within which a cell therapy product can be contained and is constructed from a first material. The connector is coupled to the container and is configured to allow fluid communication between the storage volume and an external volume. The connector is constructed from the first material. The tube is coupled to the connector and is constructed from second material different than the first material. The tube and the connector are coupled to an edge of the container to produce a substantially hermetic seal.

### CROSS-REFERENCE TO RELATED APPLICATIONS

This application claims priority to U.S. Provisional Application Ser. No. 62/722,810, entitled “Cell Storage Container for Use with Sterile Docking Systems,” filed Aug. 24, 2018, which is incorporated herein by reference in its entirety.

### BACKGROUND

The embodiments described herein relate containers for storing and handling cells and other biological materials. More particularly, the embodiments described herein relate to devices and methods including cell storage containers that can be used with conventional sterile docking equipment.

Cell therapy involves administering living cells to a patient for the treatment of a disease, repair of tissue, or other therapeutic purposes. Cell therapy has been shown to have many potential applications, including the treatment of cancer, dementia, hematologic/immunological disorders, neurological diseases, diabetes, and repairing burned tissue. The cell therapy products can be taken from the patient (i.e., autologous source) or from another individual (i.e., allogeneic source), and can be derived from stem cells. Bone marrow transplantation is one example of cell therapy.

Many known cell therapy products are developed by first harvesting or withdrawing target cells. The target cells are then then stored and/or conditioned in preparation for delivery to the patient. For example, some known cell therapy products are purified (e.g., by a centrifuge or other methods), filtered, cultured, and/or conditioned with reagents to prepare the cells for delivery to the patient. In some circumstances, the cell products are stored at low temperatures (below freezing, or even at cryogenic temperatures) for a period of time before being administered to the patient. Accordingly, cell therapy methods include the use of containers for handling, treatment, and storage of the cell products. Known cell storage containers often have at least one port (or connection) to allow cells to flow into the container and at least one port (or connector) to allow cells, media, waste, or other materials flow out of the container.

In some instances, it is desirable to use cell storage containers with known sterile docking equipment and procedures. Sterile docking is a method of fluidically coupling one or more containers within a system, for example, an intravenous system to administer substances to a patient. This process minimizes the handling and manual connection of containers. Specifically, known methods of sterile docking include cutting and joining two sterile, closed end tubes using a hot severing means while maintaining system sterility. The closed ends of the tubes are severed by a cutter that is maintained at a temperature hot enough to kill bacteria. Known sterile docking equipment also encloses the cutter and tube ends to limit the likelihood that viable bacteria will be conveyed into either of the tubes or the subsequent joint. Sterile docking is commonly used in the handling of blood or other blood-based procedures, such as dialysis procedures. Accordingly, because known blood storage bags are typically constructed from polyvinyl chloride (PVC), conventional sterile docking equipment and methods are tailored for use with PVC tube ends.

Because cell therapy and cell therapy products are subject to different conditions and have different properties than blood, conventional blood storage bags are not often the desired choice for the storage and handling of cells. For example, some known cell storage containers are constructed from non-PVC materials, for example, to provide the desired gas permeability for the desired handling (e.g., culturing, etc.). Because the attachment of dissimilar polymeric materials often presents technical challenges, such known non-PVC storage containers often do not include inlet or outlet tubes constructed from PVC. Thus, such known storage containers are incompatible with conventional sterile docking equipment and methods.

Some known storage containers include in-line connectors, such as barbed fittings, to allow for connection of dissimilar materials (i.e., a PVC tube to a non-PVC bag). The inclusion of such known connectors, however, often produces undesirable performance or flow characteristics when the cell therapy products are conveyed into or out of the container. For example, some known connectors do not produce a desired hermetic seal. Other known connectors produce undesirable expansions or contractions in the flow area, which can damage cells.

Thus, a need exists for improved containers and methods for storing and handling cells and other biological material.

### SUMMARY

Containers, methods of manufacturing containers, and methods for storing cells and other biological materials are described herein. In some embodiments, an apparatus includes a container, a connector, and a tube. The container defines a storage volume within which a cell therapy product can be contained and is constructed from a first material. The connector is coupled to the container and is configured to allow fluid communication between the storage volume and an external volume. The connector is constructed from the first material. The tube is coupled to the connector and is constructed from second material different than the first material. The tube and the connector are coupled to an edge of the container to produce a substantially hermetic seal.

In some embodiments, an end portion of the tube is coupled within the connector and an end portion of the connector is coupled to the edge of the container. In some embodiments, the end portion of the tube includes a first end surface and the end portion of the connector includes a second end surface. The second end surface being aligned with the first end surface. Additionally, the first end surface and the second end surface are substantially flush with an inner surface of the container.

In some embodiments, a method of conveying a cell therapy product includes placing a first end portion of a first tube of a container assembly into a sterile docking system. The container assembly includes a container, a connector, and the first tube. The container defines a storage volume within which the cell therapy product can be contained. The container is constructed from a first material. A second end portion of the first tube is coupled within the connector and an end portion of the connector and the second end portion of the first tube are coupled to an edge of the container to produce a substantially hermetic seal. The first tube is constructed from a second material different than the first material. An end portion of a second tube is placed into the sterile docking system. The second tube is constructed from the second material. The sterile docking system is then actuated to join the first end portion of the first tube to the end portion of the second tube. After the actuating, the cell therapy product is conveyed through the first tube and the second tube.

### BRIEF DESCRIPTION OF THE DRAWINGS

FIG. 1 is plan view of a container according to an embodiment.

FIG. 2 is a schematic illustration of a portion of a container according to an embodiment.

FIG. 3 is a flow chart of a method of manufacturing a container assembly according to an embodiment.

FIG. 4 is a flow chart of a method of conveying cells according to an embodiment.

### DETAILED DESCRIPTION

The embodiments described herein can advantageously be used in a wide variety of cell therapy applications, including handling, storage, processing, and implantation operations. In particular, the container designs described herein can be used with convention sterile docking systems to promote efficient, sterile movement of cell therapy products into and/or out of the container.

As used herein, the term “about” when used in connection with a referenced numeric indication means the referenced numeric indication plus or minus up to 10% of that referenced numeric indication. For example, the language “about 50” covers the range of 45 to 55. Similarly, the language “about 5” covers the range of 4.5 to 5.5.

As used herein, the term cell or cell therapy product refers to any material that is related to cell therapy procedures. Thus, a cell can be a stem cell, a processed (or purified) cell or cluster of cells, a cultured cell or cluster of cells.

As used in this specification, specific words chosen to describe one or more embodiments and optional elements or features are not intended to limit the invention. For example, spatially relative terms—such as “beneath”, “below”, “lower”, “above”, “upper”, “proximal”, “distal”, and the like—may be used to describe the relationship of one element or feature to another element or feature as illustrated in the figures. These spatially relative terms are intended to encompass different positions (i.e., translational placements) and orientations (i.e., rotational placements) of a device in use or operation in addition to the position and orientation shown in the figures. For example, if a device in the figures is turned over, elements described as “below” or “beneath” other elements or features would then be “above” or “over” the other elements or features. Thus, the term “below” can encompass both positions and orientations of above and below. A device may be otherwise oriented (e.g., rotated 90 degrees or at other orientations) and the spatially relative descriptors used herein interpreted accordingly. Likewise, descriptions of movement along (translation) and around (rotation) various axes includes various spatial device positions and orientations.

Similarly, geometric terms, such as “parallel”, “perpendicular”, “round”, or “square”, are not intended to require absolute mathematical precision, unless the context indicates otherwise. Instead, such geometric terms allow for variations due to manufacturing or equivalent functions. For example, if an element is described as “round” or “generally round”, a component that is not precisely circular (e.g., one that is slightly oblong or is a many-sided polygon) is still encompassed by this description.

In addition, the singular forms “a”, “an”, and “the” are intended to include the plural forms as well, unless the context indicates otherwise. The terms “comprises”, “includes”, “has”, and the like specify the presence of stated features, steps, operations, elements, components, etc. but do not preclude the presence or addition of one or more other features, steps, operations, elements, components, or groups.

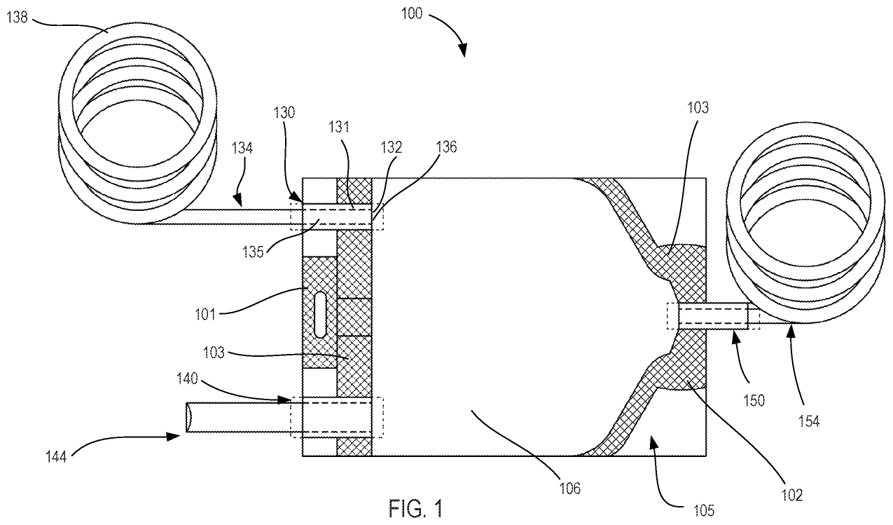

FIG. 1 illustrates a container assembly 100 according to an embodiment. The container assembly 100 (and any of the container assemblies described herein) can be used to contain cell therapy products for storage, processing, or any suitable use in connection with cell therapy procedures. The container assembly 100 includes a container 105 and three connector/tube assemblies (which can act as ingress and/or egress ports). Specifically, the connector/tube assemblies include a first connector 130 coupled to a first tube 134, a second connector 140 coupled to a second tube 144, and a third connector 150 coupled to a third tube 154. The container 105 includes a first end portion 101, a second end portion 102, and a pair of edges 103 at the first end portion 101 and the second end portion 102. The container 100 is constructed from a first layer and a second layer coupled together to define a storage volume 106.

The container 105 is constructed from a first material that is compatible with and/or facilitates the storage of cells within the storage volume. For example, in some embodiments, the first material can be a material having a low barrier polymer that can be suitable for certain cell handling operations. For example, in some embodiments, the first material can allow gaseous transfer sufficient to facilitate cell culturing operations. Examples of such low barrier materials include (but are not limited to) EVA, PTFE, PFA, FEP, or ETF. In other embodiments, the first material can be a high barrier polymer that can be suitable for other cell handing operations. Examples of such high barrier materials include (but are not limited to) LLDPE, LDPE, PET, MDPE, EAA, Nylon, or PEEK.

In some embodiments, the container, including can be constructed from any combination of such materials, including laminates constructed from multiple different materials. For example, in some embodiments, the container 105 (including the first layer and the second layer) is a laminate that includes a substrate and a barrier coating. The substrate can be, for example, a peelable film and the barrier coating can be any suitable coating, such as an aluminum oxide barrier coating of any suitable thickness (36 gauge, 40 gauge, 48 gauge, or any thickness therebetween).

The first connector 130 and first tube 134 are coupled to the first end portion 101 of the container 105 and can be in ingress through which cells or other cell-related material is transferred into the volume 106. The second connector 140 and second tube 144 are coupled to the first end portion 101 of the container 105 and can also be in ingress through which cells or other cell-related material is transferred into the volume 106. The third connector 150 and third tube 154 are coupled to the opposite second end portion 102 of the container 105 and can be in egress through which cells or other cell-related material is transferred out of the volume 106. The construction of the connectors and tubes is similar, and thus only the first connector 130 and first tube 134 are described in detail. The first connector has an end portion 131 coupled to an edge 103 of the container. In some embodiments, the end portion 131 includes an end (or terminal) surface 132 that is positioned to be substantially flush to an inner surface of the container. Said another way, the end portion 131 and/or the end surface 132 are positioned within the internal volume 106 such that the magnitude of any sudden flow expansion or any sudden flow contraction is minimized. In this manner, when cell-related materials are flowing into or out of the cell via the connector 130, there are limited surfaces extending into the flow path that can produce undesirable turbulence, eddy currents, or otherwise provide surface onto which cells can adhere. This arrangement, produces an environment to enhance cell viability.

The connector 130 is constructed from the first material—that is, the same material from which the container 105 is constructed. In this manner, the connector 130 can be coupled to the container in a robust manner. Similarly stated, the connector 130 can be coupled to the container 105 to produce a substantially or sufficiently hermetic seal for cell therapy applications. For example, in some embodiments, the connector 130 (or any of the connectors) can be heat bonded to the edge 103 of the container 105. For example, the connector can be coupled to the container via a radio frequency weld, a laser weld, and a direct heat (i.e., hot melt) weld. Because the connector is constructed from the same (or in other embodiments, a compatible) material as the container 105, such heat bonding (also referred to as a plastic weld) can produce a coupling having the desired strength, permeability, and other properties.

The first tube 134 includes a first end portion 135 and a second end portion 138. The first end portion 135 is coupled within the connector 130 such that the first end portion 135 and the end portion 131 of the connector 130 are together coupled to the edge 103 of the container 105. The first tube 134 is constructed from a second material that is different from the first material. In particular, the first tube 134 is constructed from a second material that is compatible for use with sterile docking equipment and methods. In some embodiments, the first tube 134 can be constructed from polyvinyl chloride (PVC). Thus, the second end portion 138 can be placed within a sterile docking system, as described herein, to be joined to another tube or portion of a cell therapy system.

In some embodiments, an end surface 136 of the tube 134 can be aligned with the end surface 132 of the connector 130. In this manner, the interface between the tube 134 and the connector 130 can be devoid of sharp edges, sudden flow expansions, sudden flow contractions, or the like. Moreover, in some embodiments, the end surface 136 can be positioned within the internal volume 106 such that the magnitude of any sudden flow expansion or any sudden flow contraction is minimized. Said another way, the end surface 136 (and the end surface 132) can be substantially flush to an inner wall of the container 105. In this manner, when cell-related materials are flowing into or out of the cell via the connector 130, there are limited surfaces extending into the flow path that can produce undesirable turbulence, eddy currents, or otherwise provide surface onto which cells can adhere. This arrangement produces an environment to enhance cell viability, recovery and/or removal from the container. Said another way, this arrangement produces a highly efficient removal system.

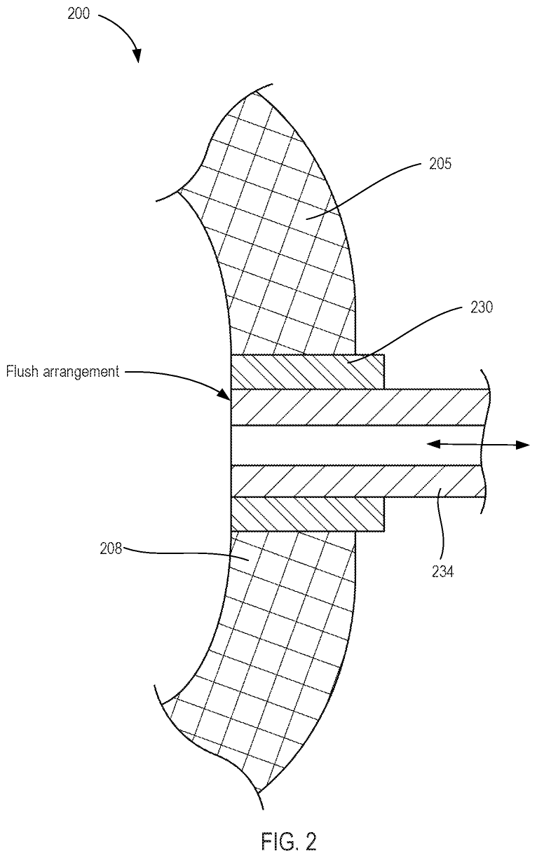

Although shown as having three connector/tube assemblies, in other embodiments, a container can include any number of ingress/egress connections. For example, FIG. 2 is a schematic illustration of a portion of an assembly 200, according to an embodiment. FIG. 2 shows a coaxial arrangement of a tube 234 and connector 230 that is coupled to a portion of a container 205 in a flush arrangement. Said another way, the end surfaces of the tube 234 and the connector 230 are aligned with (and/or form a continuous surface with) an inner surface 208 of the container 205. The first end portion of the tube 234 can be coupled within the connector 230 by any suitable mechanism to produce the desired properties (e.g., a sufficiently hermetic seal, sufficient seal strength, or the like). In some embodiments, for example, the first end portion of the tube 234 can be coupled within the connector 230 by being co-extruded with the connector, by overmolding, a glue joint, a plastic weld (i.e., thermal bond), or any other mechanism described herein.

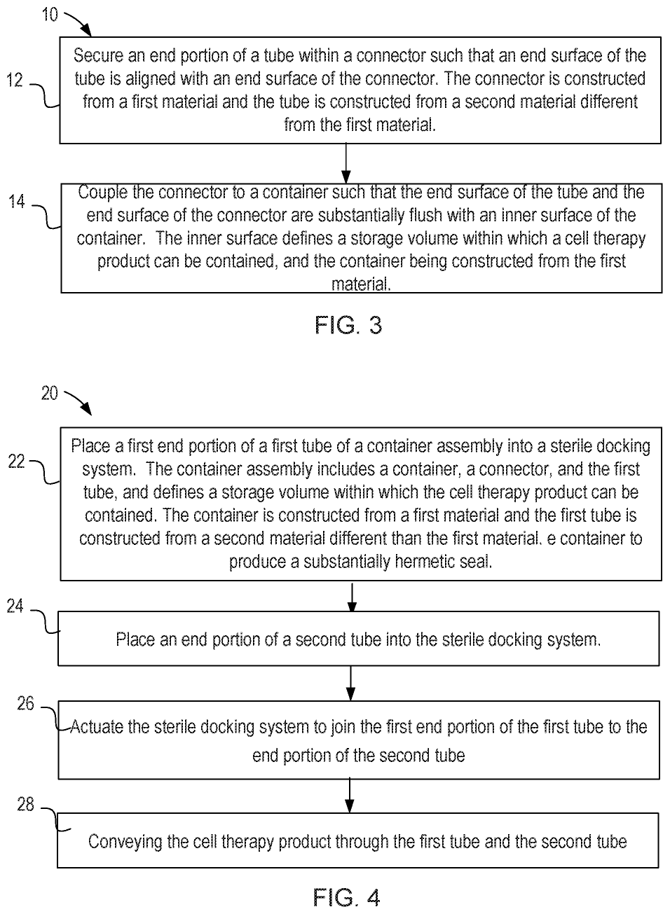

FIG. 3 is a flow chart showing a method 10 of manufacturing (or constructing) a container assembly, according to an embodiment. The method 10 can be used to construct the container assembly 100 or any other container assembly having a tube that is constructed from a dissimilar material from the container body. The method 10 includes securing an end portion of a tube within a connector such that an end surface of the tube is aligned with an end surface of the connector, at 12. The connector is constructed from a first material and the tube is constructed from a second material different from the first material. The end portion of the tube can be secured within the connector, for example, by co-extruding the tube and the connector or overmolding the connector about the tube.

The method further includes coupling the connector to a container such that the end surface of the tube and the end surface of the connector are substantially flush with an inner surface of the container, at 14. The inner surface defines a storage volume within which a cell therapy product can be contained. The container is constructed from the first material. In some embodiments, the connector can be coupled to the container by any suitable method, such as be a radio frequency weld, a laser weld, or a direct heat weld.

In some embodiments, the first material is linear low-density polyethylene (LLDPE) and the second material is polyvinyl chloride (PVC).

In some embodiments, the container assembly 100, the container assembly 200, or any suitable container assembly can be used to contain a cell therapy material. Such containers can be used in connection with a sterile docking device to convey the cell therapy materials into or out of the container assembly. For example, FIG. 4 is a flow chart showing a method 20 of conveying a cell therapy material according to an embodiment. Although the method 20 is described with reference to the container assembly 100, the method 20 can be performed with any of the container assemblies described herein or any other suitable container. As shown in FIG. 4, the method 20 includes placing a first end portion of a first tube of a container assembly into a sterile docking system, at 22. The container assembly includes a container, a connector, and the first tube, and defines a storage volume within which the cell therapy product can be contained. The container is constructed from a first material and the first tube is constructed from a second material different than the first material. In some embodiments, a second end portion of the first tube is coupled within the connector, and an end portion of the connector and the second end portion of the first tube are coupled to an edge of the container to produce a substantially hermetic seal.

In some embodiments, the second end portion of the tube includes a first end surface and the end portion of the connector includes a second end surface. The second end surface is aligned with the first end surface. In some embodiments, the first end surface and the second end surface are substantially flush with an inner surface of the container.

An end portion of a second tube is placed into the sterile docking system, at 24. In some embodiments, the second tube is constructed from the second material. The sterile docking system is then actuated to join the first end portion of the first tube to the end portion of the second tube, at 26. After the first tube is joined to the second tube, the cell therapy product is conveyed through the first tube and the second tube, at 28. The cell therapy product can be conveyed into or out of the container assembly.

While various embodiments have been described above, it should be understood that they have been presented by way of example only, and not limitation. Where methods and/or schematics described above indicate certain events and/or flow patterns occurring in certain order, the ordering of certain events and/or operations may be modified. While the embodiments have been particularly shown and described, it will be understood that various changes in form and details may be made.

For example, although the container 105 and the first connector 130 are described as being constructed from the same material to promote coupling these two components together, in other embodiments, the first connector 130 (and any of the connectors shown and described herein) can be constructed from a different, but compatible material than the container. In this manner, the connector can still be suitable (i.e., hermetically) joined to the container, and can also be coupled to the tubing, which is constructed from a different material (e.g., PVC).

Although the tube is described as being constructed from PVC, in other embodiments, the tube could be constructed from any suitable material.

Although various embodiments have been described as having particular features and/or combinations of components, other embodiments are possible having a combination of any features and/or components from any of embodiments as discussed above. Aspects have been described in the general context of medical devices, and more specifically tissue packaging devices, but inventive aspects are not necessarily limited to use in medical devices and tissue packaging.

What is claimed is:

1. An apparatus, comprising:
a container defining a storage volume within which a cell therapy product can be contained, the container constructed from a first material;
a connector coupled to the container, the connector configured to allow fluid communication between the storage volume and an external volume, the connector constructed from the first material; and
a tube coupled to the connector, the tube constructed from a second material different than the first material, the tube and the connector coupled to an edge of the container to produce a substantially hermetic seal.

2. The apparatus of claim 1, wherein:
an end portion of the tube is coupled within the connector; and
an end portion of the connector is coupled to the edge of the container.

3. The apparatus of claim 2, wherein:
the end portion of the tube includes a first end surface; and
the end portion of the connector includes a second end surface, the second end surface being aligned with the first end surface.

4. The apparatus of claim 3, wherein the first end surface and the second end surface are substantially flush with an inner surface of the container.

5. The apparatus of claim 4, wherein the end portion of the tube and the end portion of the connector are heat bonded to the edge of the container.

6. The apparatus of claim 1, wherein the first material is any one of linear low-density polyethylene (LLDPE) or of low-density polyethylene (LDPE) and the second material is polyvinyl chloride (PVC).

7. The apparatus of claim 1, wherein the tube defines a lumen through which the cell therapy product can be conveyed, the cell therapy product maintaining separation from the connector.

8. The apparatus of claim 1, wherein the container is a flexible container constructed from a first layer and a second layer.

9. The apparatus of claim 8, wherein:
the first layer is a first laminate including a first substrate and a first barrier coating; and
the second layer is a second laminate including a second substrate and a second barrier coating.

10. The apparatus of claim 8, wherein the connector is coupled to the edge of the flexible container between the first layer and the second layer.

11. A method of conveying a cell therapy product, comprising:
placing a first end portion of a first tube of a container assembly into a sterile docking system, the container assembly including a container, a connector, and the first tube, the container defining a storage volume within which the cell therapy product can be contained, the container constructed from a first material, a second end portion of the first tube coupled within the connector, an end portion of the connector and the second end portion of the first tube coupled to an edge of the container to produce a substantially hermetic seal, the first tube constructed from a second material different than the first material;
placing an end portion of a second tube into the sterile docking system, the second tube constructed from the second material;
actuating the sterile docking system to join the first end portion of the first tube to the end portion of the second tube; and
conveying, after the actuating, the cell therapy product through the first tube and the second tube.

12. The method of claim 11, wherein the first material is linear low-density polyethylene (LLDPE) and the second material is polyvinyl chloride (PVC).

13. The method of claim 12, wherein the container is a flexible container constructed from a first layer and a second layer.

14. The method of claim 13, wherein:
the first layer is a first laminate including a first substrate and a first barrier coating; and
the second layer is a second laminate including a second substrate and a second barrier coating.

15. The method of claim 14, wherein the connector is coupled to the edge of the flexible container between the first layer and the second layer.

16. The method of claim 11, wherein:
the second end portion of the tube includes a first end surface; and
the end portion of the connector includes a second end surface, the second end surface being aligned with the first end surface.

17. The method of claim 16, wherein the first end surface and the second end surface are substantially flush with an inner surface of the container.

18. A method of constructing a container assembly, comprising:
securing an end portion of a tube within a connector such that an end surface of the tube is aligned with an end surface of the connector, the connector being constructed from a first material, the tube being constructed from a second material different from the first material; and
coupling the connector to a container such that the end surface of the tube and the end surface of the connector are substantially flush with an inner surface of the container, the inner surface defining a storage volume within which a cell therapy product can be contained, the container being constructed from the first material.

19. The method of claim 18, wherein the first material is linear low-density polyethylene (LLDPE) and the second material is polyvinyl chloride (PVC).

20. The method of claim 19, wherein:
the securing the end portion of the tube includes any of co-extruding the tube and the connector or overmolding the connector about the tube; and
the coupling the connector to the container is performed by any of a radio frequency weld, a laser weld, or a direct heat weld.
